(19) United States
(12) Patent Application Publication
(10) Pub. No.: US 2007/0100247 A1
(43) Pub. Date: May 3, 2007

(54) Combined wrist blood pressure and ecg monitor

(76) Inventors: Harry Louis Platt, East Gardens (AU); Allan Michael Shell, East Gardens (AU); Vladimir Jankov, East Gardens (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/536,605

(22) PCT Filed: Nov. 25, 2003
(86) PCT No.: PCT/AU03/01568

§ 371 Date: Nov. 30, 2006

(30) Foreign Application Priority Data
Nov. 25, 2002 (AU) 2002952927

(51) Int. Cl. A61B 5/04; A61B 5/02

(52) U.S. Cl. 600/513; 600/490

(57) ABSTRACT

A physiological monitor device (10) has physiological detection means, signal transducer means, control and calculating means as well as a display (15). The detection means has an inflatable cuff (12) with a pressure detection for blood pressure and an ECG electrode secured to the cuff. The cuff fits about the wrist of the user with the control, calculating and display included in a module (11) attached to the cuff.

### FIELD OF THE INVENTION

The present invention relates generally to diagnostic medical devices and, in particular to a device worn on a user's wrist which measures a number of biological parameters.

### BACKGROUND TO THE INVENTION

Portable diagnostic medical devices are used for monitoring of biological signals of patients in order to detect disease.

Regular monitoring of blood pressure is recommended to patients with hypertension. Digital blood pressure monitors are accurate and convenient for home use. These monitors are easy to operate and they offer electronic data storage. Some of these monitors have a capability for the remote data transfer that is used for analysis of the dynamic of blood pressure-changes by the medical practitioner.

Patients with hypertension also have higher risk of heart diseases. A number of portable ECG recorders for ambulatory use are available for heart disease diagnostic. These devices are capable of electronic storage of user's electrocardiogram and of remote transmission of the stored signals for analysis by the medical practitioner. Portable ECG recorders are highly effective in detection of heart rhythm disorders.

It would be an advantage to monitor simultaneously both blood pressure and electrocardiogram in the patients with hypertension using single device.

### OBJECT OF THE INVENTION

It is an object of present invention to provide a method and apparatus for simultaneous monitoring of blood pressure and electrocardiogram using an electronic device that incorporates blood pressure monitor and electrocardiograph

It is also an object of present invention to provide means for remote transmission of measured data in the above-mentioned device.

### DISCLOSURE OF THE INVENTION

According to one aspect of the present invention there is disclosed a physiological monitor device having physiological detection means, signal transducer means, control and calculating means, display means, wherein said detection means includes an inflatable cuff means with pressure detection means to test for blood pressure and ECG electrode means to test for ECG, said electrodes being adapted to be secured at least to said cuff means.

Preferably, the cuff means is adapted to be secured to the wrist of a user, with at least some of the electrodes coming into contact with the skin of the user when so secured.

Preferably, the remainder of the electrodes are adapted to be either held by the user or attached to the user's body.

Preferably, the device monitors blood pressure and ECG measurements simultaneously.

### BRIEF DESCRIPTION OF THE DRAWINGS

One embodiment of the invention will now be described with reference to the accompanying drawings in which:

FIG. 1 is a perspective view of a wrist blood pressure monitor,

FIG. 2 is a block diagram of the blood pressure monitor;

FIG. 3 is a block diagram of a typical single lead ECG recorder;

FIG. 4 is a block diagram of blood pressure monitor with ECG recorder function; and

FIG. 5 is perspective view of a wrist blood pressure monitor with ECG recorder functions of a preferred embodiment.

### BEST MODE OF CARRYING OUT THE INVENTION

In the preferred embodiment of present invention a portable digital blood pressure monitor with ECG module is described.

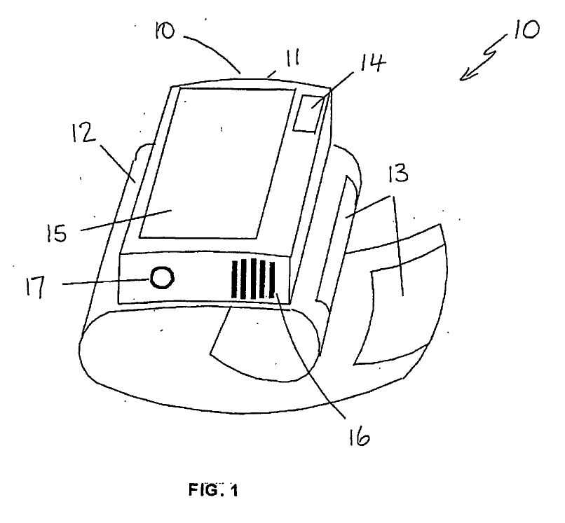

Referring to FIG. 1, a portable blood pressure monitor is shown. Wrist blood pressure monitor 10 has a device enclosure 11 and inflatable cuff 12. The cuff 12 is placed around the user's wrist and secured by two Velcro pads 13. The wrist blood pressure monitor 10 is turned on by button 14. A typical wrist blood pressure monitor is capable of automatic inflation of the cuff, termination of inflation when a predetermined cuff pressure level is reached and measurement of systolic and diastolic blood pressure and heart rate. Measured values are displayed on an LCD display 15.

Referring again to FIG. 1, a mini-speaker output 16 is shown. The speaker is used to produce audio tones when the user's heartbeat is detected or to generate warning sounds when measurement errors occur.

Referring again to FIG. 1, a communication connector 17 is shown. This connector 17 is used for connection to a PC (not illustrated) for reading blood pressure data stored in the device 10.

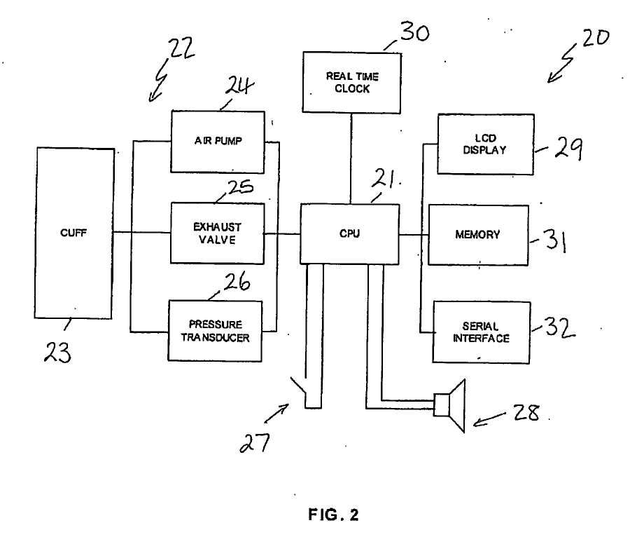

A functional block-diagram of a digital blood pressure monitor (BPM) 20 is shown in FIG. 2. The core of the BPM is a CPU unit 21. The CPU 21 of the BPM 20 controls all peripherals of the device 20 and performs calculations necessary for blood pressure determination.

A pneumatic circuit 22 of the BPM 20 is shown in FIG. 2. The pneumatic circuit 22 is formed by a cuff 23, an air pump 24, an exhaust valve 25 and a pressure transducer 26. Peripherals of the CPU 21 are shown in FIG. 2 and include on button, 27, speaker 28, LCD display 29, real time clock 30, memory 31 and serial interface 32.

The BPM 20 is operated such that when button 27 is depressed by the user, the CPU 21 measures cuff air pressure from the pressure transducer 26 and calibrates ‘zero’ pressure. The CPU 21 then turns on normally opened valve 25 and the air pump 24. The air pump 24 creates pressure in the cuff 23. The CPU 21 via the pressure transducer 26 continually monitors the cuff pressure. When the cuff pressure reaches a predetermined level, the CPU 21 stops the pump 24 and starts to measure blood pressure. During measurement the CPU 21 detects heart beats and generates short audio beeps through a speaker 28. When blood pressure is measured, the CPU 21 turns off valve 25 and displays the measured values on a LCD display 29. The CPU 21 reads current time from real time clock 30 and stores measured blood pressure values, heart rate and date and time of the measurement into a memory 31. Once stored in the memory 31 data can be transmitted to the PC via serial interface 32 or by generating modulated audio tones via the speaker 28,

Such operation of digital blood meters is well known within the art.

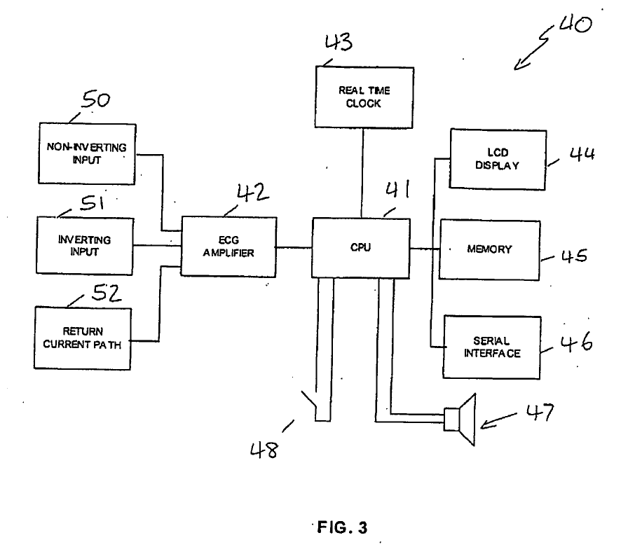

Referring to FIG. 3, a functional block diagram of typical single lead ECG recorder 40 is shown. A CPU 41 controls peripherals of the ECG recorder 40. The peripherals include ECG amplifier 42, real time clock 43, LCD display 44, data memory 45, serial interface 46, speaker 47, push button ‘Record’ 48 and push button ‘Transmit’ 49.

The CPU 41 of ECG recorder 40 receives ECG signals via non-inverting input 50, inverting input 51 and current return path 52 through the ECG amplifier 42. The ECG amplifier 42 amplifies the ECG signal to a level acceptable by the requirements of dynamic range of the ECG recorder 40.

The non-inverting input 50, inverting input 51 and current return path 52 are electrically connected to three external ECG electrodes (not illustrated). The ECG electrodes can be used in form of adhesive press-stud electrodes connected to the three-wire cable or in form of conductive pads located on the enclosure of the ECG recorder. A combination of cable electrodes and conductive electrodes can also be used. The ECG electrodes are applied to the skin of the patient when the ECG signal is acquired.

In its operation, when button 48 ‘Record’ is depressed, the CPU 41 reads current time from the real time clock 43 and starts to acquire signal from the ECG amplifier 53 and record it into data memory 45. Date and time of the beginning of the recording is stored in the particular location of the data memory 45. The CPU 41 displays the ECG signal on the LCD display 44. The ECG recorder 40 typically acquires and records data from several seconds to several hours. When recording is finished, the CPU 41 issues an ‘End of recording’ audio tone via the speaker 47. When button 49 ‘Transmit’ is depressed, the CPU 41 transmits ECG data stored in the memory 45 via the serial interface 46 and/or using modulated audio tones via speaker 47.

It can be seen that functional block-diagrams of the blood pressure monitor and ECG recorder are pretty similar. Both of them have the same peripherals except for the source of the signal and two pneumatic components. It would be an advantage to share common for both devices peripherals in order to minimize costs and combine their functionality.

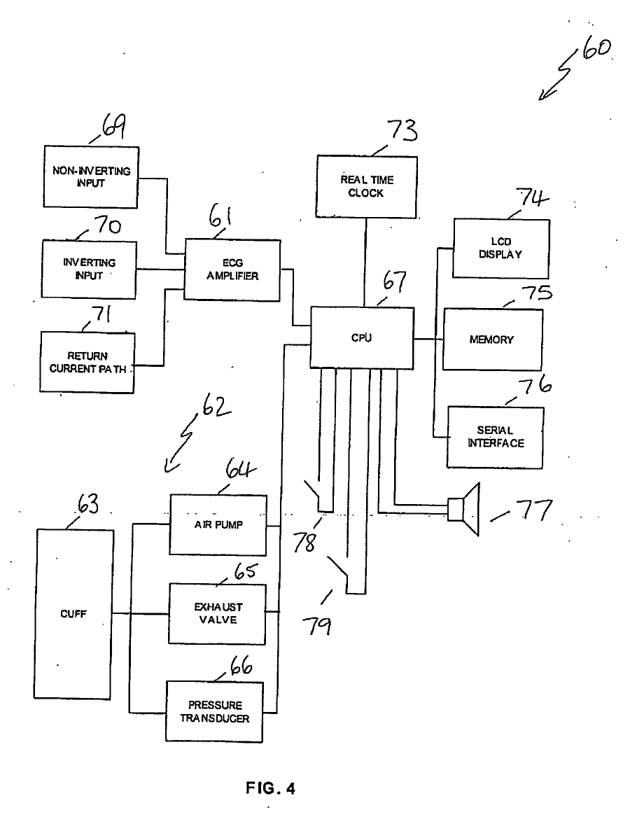

A functional block-diagram of digital blood pressure monitor with ECG-recorder (BPM-ECG) 60 is shown in FIG. 4.

Referring again to FIG. 2, FIG. 3 and FIG. 4, all three functional block diagrams include CPU, real time clock, LCD display, data memory, serial interface, speaker and button(s).

Referring again to FIG. 4, functional block-diagram of BPM-ECG 60 includes ECG amplifier 61 and pneumatic circuit 62. The pneumatic circuit 62 includes an inflatable cuff 63, an air pump 64, a valve 65 and a pressure transducer 66. The BPM-ECG 60 further includes a CPU 67 which is connected to the output of the ECG amplifier 61 which is connected to via non-inverting input 69, inverting input 70 and current return path 71; and to the components of pneumatic circuit 62.

The BPM-ECG 60 further includes peripherals which include real time clock 73, LCD display 74, data memory 75, serial interface 76, speaker 77, push button ‘Record’ 78 and push button ‘Transmit’ 79.

Referring again to FIG. 4, when button 78 ‘Record’ is depressed, the CPU 67 calibrates its pressure transducer 66, turns on valve 65 and air pump 64. At the same time the CPU 67 receives ECG signals from electrodes associated with the non-inverting input 69, the inverting input 70 and the current return path 71 via the amplifier 61. The CPU 67 records ECG signal into the memory 75 and displays it on the LCD 74.

When measurement of blood pressure is finished, the CPU 67 stops recording and display of the ECG signal and displays measured blood pressure values. Then the CPU 67 reads real time clock 73 and stores date, time and measured values in the data memory 75.

Referring again to FIG. 4, when button 79 ‘Transmit’ is depressed, the CPU 67 reads stored ECG and blood pressure data from its memory 75 via serial interface 76 and/or using modulated audio tones via speaker 77.

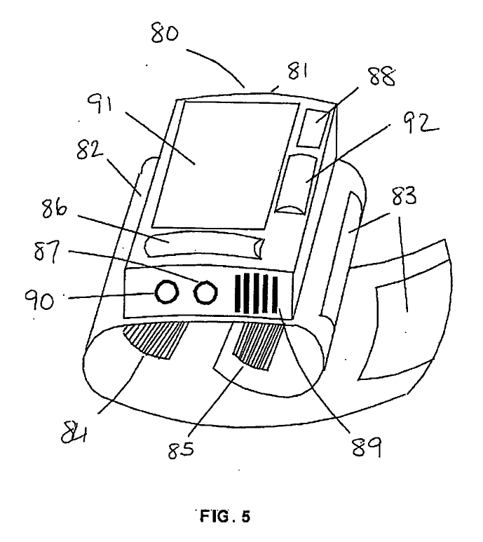

Referring to FIG. 5, a drawing of the preferred embodiment BPM-ECG 80 is shown. The BPM-ECG 80 includes enclosure 81, inflatable cuff 82 with Velcro pads 83. On the inner surface of the cuff 83, two conductive ECG electrodes 84 (non-inverting input) and 85 (current return path) are positioned. The third, inverting input of ECG amplifier is connected to an external pad electrode 86 and to a one-way connector 87. When the BPM-ECG 80 is placed around the user's wrist, electrodes 84 and 85 come in close contact with the skin. The external electrode 85 is exposed and the user can touch it with the finger to provide connection to the inverting input of the ECG amplifier. Alternatively, a single wire lead with adhesive press-stud electrode can be placed to the chest of the user and connected via connector 87 to the inverting input of ECG amplifier. An on button 88, mini output speaker 89, connector 90, display 91 and record button 92 are used in the aforementioned manner.

Throughout the specification, the word “comprise” and its derivatives are intended to have an inclusive rather than an exclusive meaning unless the context requires otherwise.

The foregoing describes only some embodiments of the present invention, and modifications obvious to those skilled in the art can be made thereto without departing from the scope of the present invention.

1. A physiological monitor device having physiological detection means, signal transducer means, control and calculating means, display means, wherein said detection means includes an inflatable cuff means with pressure detection means to test for blood pressure and ECG electrode means to test for ECG, said electrodes being adapted to be secured at least to said cuff means.

2. The physiological monitor device according to claim 1, wherein the cuff means is adapted to be secured to the wrist of a user, with at least some of the electrodes coming into contact with the skin of the user when so secured.

3. The physiological monitor device according to claim 2, wherein the remainder of the electrodes are adapted to be either held by the user or attached to the user's body.

4. The physiological monitor device according to claim 1, wherein the device monitors blood pressure and ECG measurements simultaneously.

5. The physiological monitor device according to claim 1, wherein measured values are stored in the device and are displayed on an LCD display means.

6. The physiological monitor device according to claim 5, wherein a speaker output is used to produce audio tones when the user's heartbeat is detected or to generate warning sounds when measurement errors occur.

7. The physiological monitor device according to claim 6, wherein a communication connector is used for connection to a computer means for reading blood pressure data stored in the device.

8. The physiological monitor device according to claim 7, wherein a CPU unit controls peripherals of the device and performs calculations necessary for blood pressure determination., the peripherals including the speaker, LCD display, real time clock, memory and serial interface.

9. The physiological monitor device according to claim 8, wherein a pneumatic circuit is formed by the cuff means, an air pump, an exhaust valve and a pressure transducer.

10. The physiological monitor device according to claim 9, wherein the blood pressure monitor is operated such that when a start button 27 is depressed by the user.

11. A physiological monitor device being substantially as described with reference to the accompanying drawings.
